(19) United States
(12) Patent Application Publication
(10) Pub. No.: US 2020/0107952 A1
(43) Pub. Date: Apr. 9, 2020

(54) DISPOSABLE, WRIST SECURING DEVICE FOR USE IN THE EMERGENCY MEDICAL FIELD

(71) Applicant: Tracy Alan White, Cocoa, FL (US)

(72) Inventor: Tracy Alan White, Cocoa, FL (US)

(21) Appl. No.: 16/596,107

(22) Filed: Oct. 8, 2019

Related U.S. Application Data
(60) Provisional application No. 62/742,548, filed on Oct. 8, 2018.

(51) Int. Cl. A61F 5/37

(52) U.S. Cl. CPC A61F 5/3723

(57) ABSTRACT

A wrist-securing device is provided. The wrist-securing device has two coextensive straps joined together along a midportion so that the resulting four end portions may pivot about the midportion. The joined midportion defines two opposing ends, a first end and a second end, each end having upper and lower straps movable relative to each other. The upper and lower straps of each end can wrap about the upper and lower surfaces, respectively, of the wrist of human wearer. Each upper and lower strap provides upper and lower fasteners to removably connect so as to secure the upper and lower straps wrapped about the wrist. Thus, each end of the wrist-securing device may secure each of the wrists of an unresponsive patient receiving emergency medical services in such a manner as to not harm the patient or occlude their distal circulation.

### CROSS-REFERENCE TO RELATED APPLICATION

This application claims the benefit of priority of U.S. provisional application No. 62/742,548, filed 8 Oct. 2018, the contents of which are herein incorporated by reference.

### BACKGROUND OF THE INVENTION

The present invention relates to emergency medical service devices and, more particularly, to a disposable, wrist-securing device for use in the emergency medical field.

Emergency medical services (EMS), also known as ambulance services or paramedic services, provide an urgent medical response, providing out-of-hospital treatment and transport to hospitals and other institutional care facilities. Being “first responders”, EMS personnel are on the front lines of medical emergencies that include treatments of acute illnesses and injuries, as well as treating those suffering from severe mental health and/or drug-related issues.

As a result, many EMS patients can be uncooperative and/or unresponsive. The upper extremities of such patients are currently not secured while the patient is in a supine position during EMS care, creating an unsafe situation for both patient and care providers. In short, currently there is no known medically safe device to effectively address and remedy this challenge.

As can be seen, there is a need for a disposable, wrist-securing device for use in the emergency medical field. The device embodied by the present invention (colloquially known as ‘Kwik-Cuff’) is adapted to securely hold the upper extremities of an unresponsive, supine patient in a safe position without inflicting harm or restraining them against their will, while providing a measure of safety to patient and EMS personnel during movement of the patient. The present invention secures the wrists of an unresponsive patient across the lower abdomen without injury to the patient, unlike current restraints. Moreover, the present invention does not occlude distal circulation. Kwik Cuff's simple design makes it easy to store prior to use, employ during use, and is disposable after a single use, preventing subsequent bodily fluid contamination.

### SUMMARY OF THE INVENTION

In one aspect of the present invention, wrist-securing device includes the following: an upper strap; a lower strap generally coextensive with the upper strap; each strap having a first end portion and an opposing second end portion; a joining element joining the upper and lower straps between their respective end portions so that said end portions are movable about the joining element; a fastener pad along a top surface of the first and second end portions of the upper strap; a fastener tab extending from the first and second end portions of the lower strap; and the fastener pads and fastener tabs adapted to removably connect so as to secure their respective end portions together, wherein each fastener tab is mutually inclusive of the upper end portion, wherein each fastener tab extends beyond a peripheral boundary of the lower end portion, and wherein each fastener tab is disposed along a bottom surface of the lower end portion.

In another aspect of the present invention, a method of securing the wrists of an unresponsive patient in an emergency medical service situation includes the following: providing the above-mentioned wrist-securing device; wrapping the upper end portion and the lower end portion of the first end portions about opposing surfaces a first wrist of said patient; wrapping the upper end portion and the lower end portion of the second end portions about opposing surfaces a second wrist of said patient; securing the fastener tab to the fastener pad for the first and second end portions; and resting said wrists across a lower abdomen of said patient.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention, shown in use;

FIG. 2 is a top perspective view of an exemplary embodiment of the present invention;

FIG. 3 is a bottom perspective view of an exemplary embodiment of the present invention;

FIG. 4 is a section view of an exemplary embodiment of the present invention, taken along line 4-4 of FIG. 2;

FIG. 5 is a section view of an exemplary embodiment of the present invention, taken along line 5-5 of FIG. 1; and

FIG. 6 is a perspective view of an exemplary embodiment of the present invention, shown in a stored condition.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a wrist-securing device formed from two coextensive straps joined together along a midportion so that the resulting four end portions may pivot about the midportion. The joined midportion defines two opposing ends, a first end and a second end, having upper and lower straps movable relative to each other. The upper and lower straps can wrap about the upper and lower surfaces, respectively, of the wrist of human wearer. Each upper and lower strap provides upper and lower fasteners, respectively, that are adapted to removably connect so as to secure the upper and lower straps wrapped about the wrist. Thus, each end of the wrist-securing device may secure each of the wrists of an unresponsive patient receiving emergency medical services in such a manner as to not harm the patient or occlude their distal circulation.

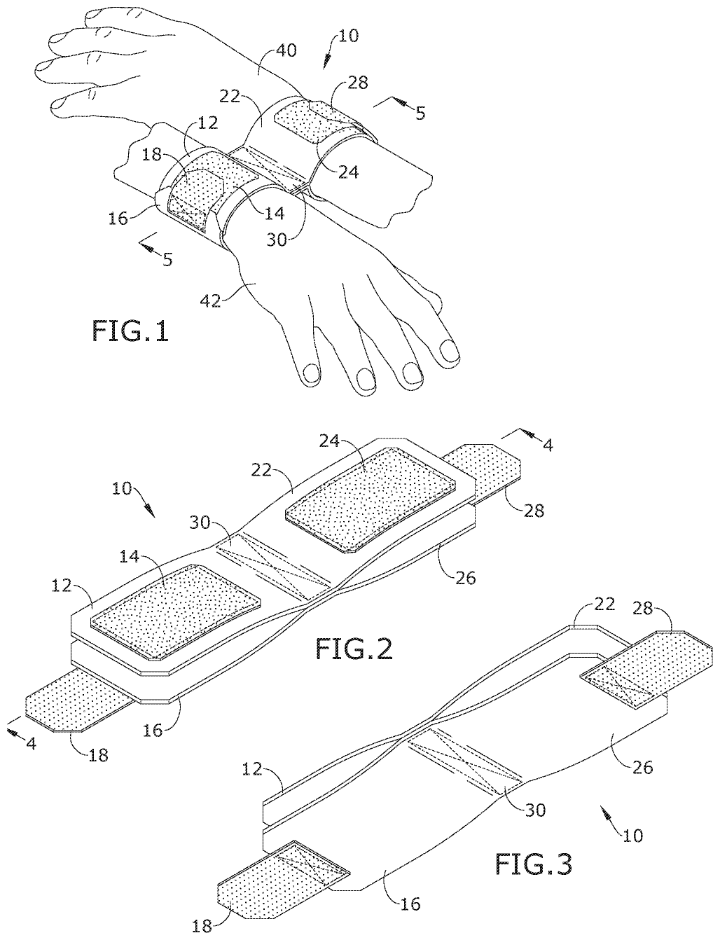

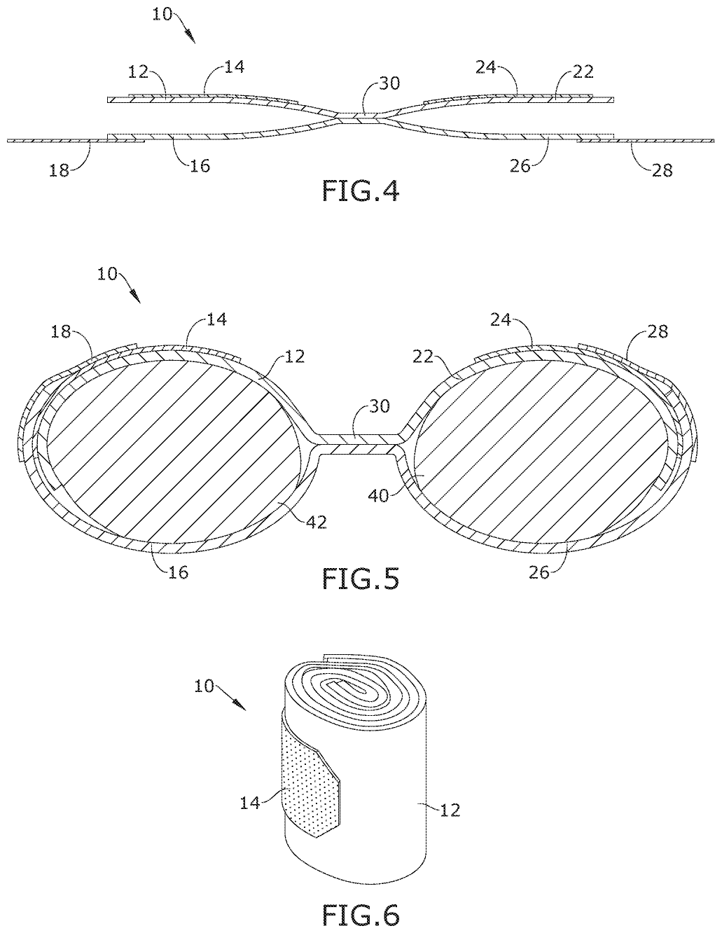

Referring to FIGS. 1 through 6, the present invention may include a wrist-securing device 10 adapted for the emergency medical services field, as well as having utility in other fields. The wrist-securing device 10 may include generally coextensive upper and lower straps that are joined at a midportion by a joining element 30. The joining element 30 may be high-strength threading, binding or other joining fasteners that rigidly connect one object to another object. Here these objects are the upper and lower straps.

In certain embodiments, the joining element 30 may be cotton/polyester sewing thread. The upper and lower straps may be soft, breathable fabric having sufficient strength and tear-resistance so as to be exposed to the stress and strain as contemplated by the use disclosed herein without failing.

As a result of the bisecting joining element 30 the upper and lower straps can be defined by a first upper strap 12, a second upper strap 22, a first lower strap 16 and a second lower strap 26. In an open condition, as illustrated in FIGS. 2 through 4, the wrist-securing device 10 takes an open-book configuration wherein the lower straps 16 and 26 act as the book cover, if you will, and the upper straps 12 and 22, the book pages. The outside of the ‘book cover’ provides a lower fastener tab 18 and 28 for the first and second lower straps 16 and 26, respectively. The lower fastener tab 18 extends beyond a distal end of each respective lower strap 16 or 26. Each ‘readable book page’ provides an upper fastener pad 14 and 24 that is mutually inclusive of each first and second upper strap 12 and 22, respectively.

In other words, the first and second upper straps 12 and 22 each provide one upper fastener pad 14/24 along an upward-facing or top surface, wherein each upper fastener pad 14/24 is mutually inclusive of their respective upper strap 12 or 22. While, the lower straps 16 and 26 provide lower fastener tabs 18 and 28, respectively along a bottom or downward-facing surface, wherein the lower fastener tabs 18/28 extend beyond a distal (relative to the midportion joining element 30) boundary of their respective lower straps 16 or 26.

It should be understood by those skilled in the art that the use of directional terms such as upper, lower, upward, downward, top, bottom, and the like are used in relation to the illustrative embodiments as they are depicted in the figures, specifically FIGS. 2 through 4. The upward direction (or upper, top) being toward the top of the corresponding figures and a downward (or lower, bottom) direction being toward the bottom of the corresponding figures.

The upper fastener pads 14/24 and lower fastener tabs 18/28 may be hook and loop fasteners or other mating fasteners that removably connect to each other.

As a result of the joining element 30 seated in the middle of the upper and lower straps, the first upper and lower straps 12 and 16 can separately wrap about the front and/or rears sides, respectively, of a first wrist 42 of a human wearer so as to overlap on the other side of said first wrist 42, whereat the associated first fastener tab 18 can overlap and operatively associate with the first fastener pad 14, as illustrated in FIG. 5. Likewise, the second upper and lower straps 22 and 26 can similarly circumscribe the second wrist 40 of the human wearer. Thus, two secured cuffs can be formed from the present invention to hold the supine patient's arm across their lower abdomen with the use of the fastener tabs and pads.

A method of using the present invention may include the following. The wrist-securing device 10 disclosed above may be provide. During emergency medical care of an unresponsive or uncooperative patient, EMS care providers would secure each wrist of that patient in one of the two cuffs provided by the present invention. In this cuffed condition, the arms of the patient may be rested across their own lower abdomen. Subsequent to its removal, the wrist-securing device 10 may be disposed of in a proper medical waste container. An amputee patient would also see benefit as the open cuff not around the patient's wrist could be attached to a backboard strap to secure the remaining extremity from dangling over side of back board. Prior to use, the wrist-securing device 10 may be rolled into a storage condition, as illustrated in FIG. 6, wherein one of the fastener tabs can removably engage one of the fastener pads.

It should be understood, of course, that the foregoing relates to exemplary embodiments of the invention and that modifications may be made without departing from the spirit and scope of the invention as set forth in the following claims.

What is claimed is:

1. A wrist-securing device, comprising:
an upper strap;
a lower strap generally coextensive with the upper strap;
each strap having a first end portion and an opposing second end portion;
a joining element joining the upper and lower straps between their respective end portions so that said end portions are movable about the joining element;
a fastener pad along a top surface of the first and second end portions of the upper strap;
a fastener tab extending from the first and second end portions of the lower strap; and
the fastener pads and fastener tabs adapted to removably connect so as to secure their respective end portions together.

2. The wrist-securing device of claim 1, wherein each fastener tab is mutually inclusive of the upper end portion, and wherein each fastener tab extends beyond a peripheral boundary of the lower end portion.

3. The wrist-securing device of claim 2, wherein each fastener tab is disposed along a bottom surface of the lower end portion.

4. The wrist-securing device of claim 1, wherein the joining element is thread.

5. The wrist-securing device of claim 1, wherein the fastener tabs and pads are hook and loop fasteners, respectively.

6. A method of securing the wrists of an unresponsive patient in an emergency medical service situation, comprising:
providing the wrist-securing device of claim 3;
wrapping the upper end portion and the lower end portion of the first end portions about opposing surfaces a first wrist of said patient;
wrapping the upper end portion and the lower end portion of the second end portions about opposing surfaces a second wrist of said patient;
securing the fastener tab to the fastener pad for the first and second end portions; and
resting said wrists across a lower abdomen of said patient.
